(19) United States
(12) Patent Application Publication
(10) Pub. No.: US 2007/0038264 A1
(43) Pub. Date: Feb. 15, 2007

(54) Methods and systems for treating autism

(76) Inventors: Kristen N. Jaax, Saugus, CA (US); Todd K. Whitehurst, Santa Clarita, CA (US); Rafael Carbunaru, Studio City, CA (US); Allison M. Foster, Los Angeles, CA (US)

Correspondence Address: STEVEN L. NICHOLS;RADER, FISHMAN & GRAVER PLLC, 10653 S. RIVER FRONT PARKWAY, SUITE 150, SOUTH JORDAN, UT, 84095, US

(21) Appl. No.: 11/315,781

(22) Filed: Dec. 21, 2005

Related U.S. Application Data
(60) Provisional application No. 60/638,608, filed on Dec. 21, 2004.

(51) Int. Cl. A61N 1/36

(52) U.S. Cl. 607/45

(57) ABSTRACT

Methods of treating autism include applying at least one stimulus to a stimulation site within the brain of a patient with an implanted stimulator in accordance with one or more stimulation parameters. Systems for treating autism include a stimulator configured to apply at least one stimulus to a stimulation site within the brain of a patient in accordance with one or more stimulation parameters.

### RELATED APPLICATIONS

The present application claims the priority under 35 U.S.C. §119(e) of previous U.S. Provisional Patent Application No. 60/638,608, filed Dec. 21, 2004, which is incorporated herein by reference in its entirety.

### BACKGROUND

Autism is a disabling neurological disorder that affects thousands of Americans and encompasses a number of subtypes. There are various putative causes of autism, but few ameliorative treatments. Autism may be present at birth, or it may develop at a later age usually early in life, for example, at ages two or three.

Autism is defined behaviorally because there are no definitive biological markers of the disorder. Behavioral symptoms of autism include abnormal development of social skills (e.g., withdrawal, lack of interest in peers, etc.), sensorimotor deficits (e.g., inconsistent responses to stimuli), and limitations in use of interactive language including both speech and nonverbal communication. Additional impairments often seen in autism include echolalia, poor symbolic thinking, a lack of imagination, self stimulation, and self injury behaviors. Disorders that often accompany autism include attention disorders, seizure disorders, Tourette's syndrome, tuberous sclerosis, mental retardation, mood disorders, depression, and other psychiatric disorders.

A limited number of treatments for autism have been developed. However, most of the treatments address the symptoms of the disease instead of the causes. For example, therapies ranging from psychoanalysis to psychopharmacology have been employed in the treatment of autism. Although some clinical symptoms may be lessened by these treatments, substantial improvement has been demonstrated in very few autistic patients. Only a small percentage of autistic persons are able to function as self-sufficient adults.

Various regions in the brain have been shown to demonstrate structural or functional abnormalities in connection with a diagnosis of autism. For example, numerous imaging studies have demonstrated increased brain size and volume in autistic patients, consistent with head circumference and postmortem studies. Studies examining regional variations suggest significant enlargements in the temporal, parietal, and occipital lobes. Other areas of the brain including, but not limited to, the fusiform gyrus, amygdala, cingulate gyrus, basal ganglia, and corpus callosum have all been shown to be enlarged or to demonstrate decreased or abnormally low activity in autistic patients.

### SUMMARY

Methods of treating autism include applying at least one stimulus to a stimulation site within the brain of a patient with an implanted stimulator in accordance with one or more stimulation parameters.

Systems for treating autism include a stimulator configured to apply at least one stimulus to a stimulation site within the brain of a patient in accordance with one or more stimulation parameters.

### BRIEF DESCRIPTION OF THE DRAWINGS

The accompanying drawings illustrate various embodiments of the present invention and are a part of the specification. The illustrated embodiments are merely examples of the present invention and do not limit the scope of the invention.

FIG. 1A depicts the lateral surface of the brain.

FIG. 1B depicts, in perspective view, the structures of the brain that make up the limbic system.

FIG. 1C is a coronal section view of the brain taken along the line indicated in FIG. 1B.

FIG. 1D illustrates an exemplary neuron.

FIG. 2 illustrates an exemplary stimulator that may be used to apply a stimulus to a stimulation site within the brain of a patient to treat autism according to principles described herein.

FIG. 3 illustrates an exemplary microstimulator that may be used as the stimulator according to principles described herein.

FIG. 4 shows one or more catheters coupled to a microstimulator according to principles described herein.

FIG. 5 depicts a number of stimulators configured to communicate with each other and/or with one or more external devices according to principles described herein.

FIG. 6 illustrates a stimulator that has been implanted beneath the scalp of a patient to stimulate a stimulation site within the brain associated with autism according to principles described herein.

Throughout the drawings, identical reference numbers designate similar, but not necessarily identical, elements.

### DETAILED DESCRIPTION

Methods and systems for treating autism are described herein. An implanted stimulator is configured to apply at least one stimulus to a stimulation site within the brain of a patient in accordance with one or more stimulation parameters. The stimulus is configured to treat autism and may include electrical stimulation, drug stimulation, gene infusion, chemical stimulation, thermal stimulation, electromagnetic stimulation, mechanical stimulation, and/or any other suitable stimulation.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

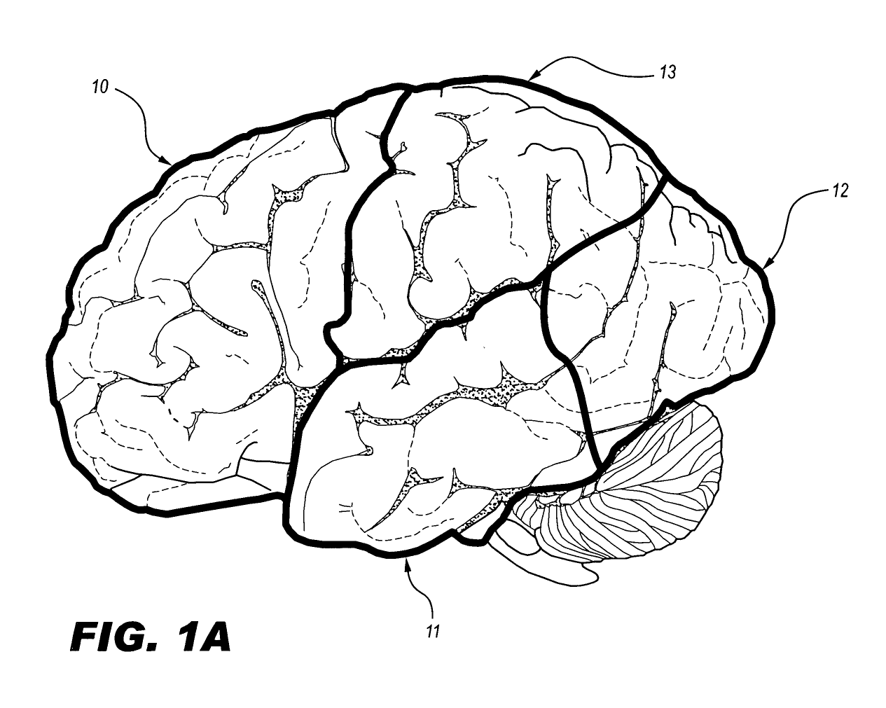

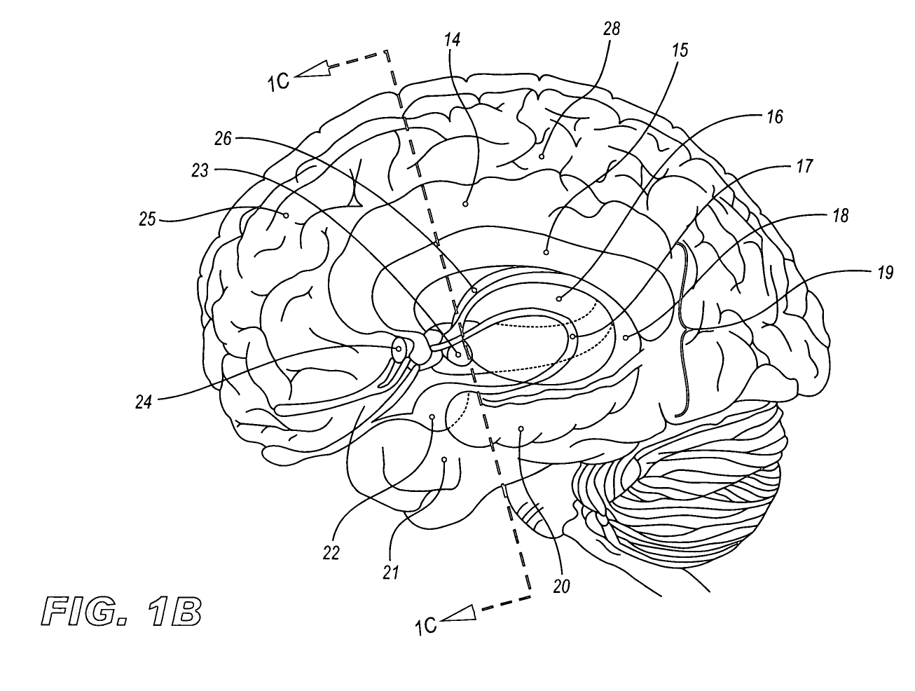

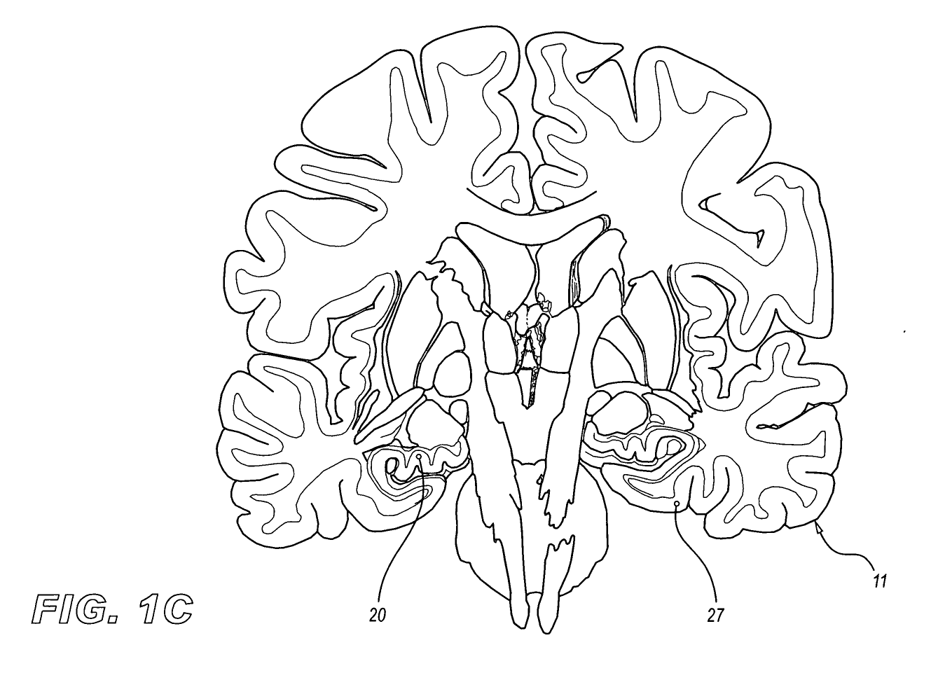

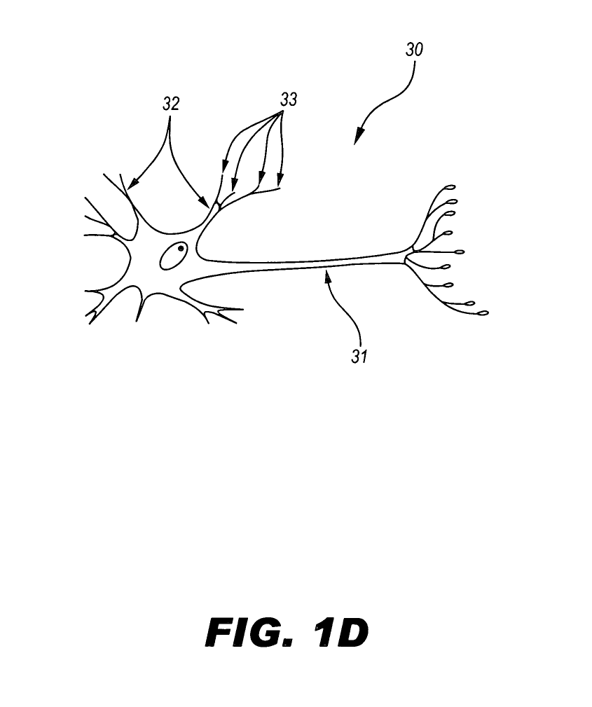

FIG. 1A depicts the lateral surface of the brain. As shown in FIG. 1A, the brain may be divided into a number of geographical lobes. The frontal lobe (10) is located at the front of the brain, the temporal lobes (11) are located on the sides of the brain, the occipital lobe (12) is located at the back of the brain, and the parietal lobe (13) is located at the top, back half of the brain. Each lobe contains areas responsible for a number of different functions.

FIG. 1B depicts, in perspective view, the structures of the brain that make up the limbic system. The limbic system is involved with emotion formation, learning, and memory. As shown in FIG. 1B, the limbic system includes, but is not limited to, several subcortical structures located around the thalamus (16). Exemplary structures of the limbic system include the cingulate gyrus (14), corpus collosum (15), thalamus (16), stria terminalis (17), caudate nucleus (18), basal ganglia (19), hippocampus (20), entorhinal cortex (21), amygdala (22), mammillary body (23), medial septal nucleus (24), prefrontal cortex (25), and fornix (26).

FIG. 1C is a coronal section view of the brain taken along the line indicated in FIG. 1B. FIG. 1C shows the hippocampus (20) and the fusiform gyrus (27). The fusiform gyrus (27) is part of the temporal lobe (11) and is involved in the processing of color information, face recognition, word recognition, and number recognition.

The brain also includes millions of neurons that innervate its various parts. FIG. 1D illustrates an exemplary neuron (30). As shown in FIG. 1D, a neuron (30) includes an axon (31) and a number of dendrites (32). The axon (31) is the long, thread-like part of the nerve cell that extends from the cell body and is configured to transmit nerve impulses to other neurons or to other structures within the patient (e.g., various portions of the brain). Dendrites (32) are the tree-like extensions of the neuron (30), as illustrated in FIG. 1D, and are configured to form synaptic contacts (33) with the terminals of other nerve cells to allow nerve impulses to be transmitted.

Synaptic contacts (33), also called synapses, are specialized junctions through which neurons signal to one another and to non-neuronal cells, such as the various areas in the brain as described in connection with FIGS. 1A-1C. Synapses (33) allow neurons to form interconnected neural circuits. They are thus vital to the biological computations that underlie perception and thought. They also allow the nervous system to connect to and control the other systems of the body. Synapses (33) that are no longer used as a person develops are normally removed by the person's nervous system—a process know as neural pruning.

Nearly every brain area has been implicated in autism. However, studies have shown that structures of the temporal lobe (11) (e.g., the fusiform gyrus (27)) and the limbic system (e.g., the cingulate gyrus (14), corpus collosum (15), thalamus (16), stria terminalis (17), caudate nucleus (18), basal ganglia (19), hippocampus (20), entorhinal cortex (21), amygdala (22), mammillary body (23), medial septal nucleus (24), prefrontal cortex (25), and fornix (26)) are most likely to be primarily responsible for the deficits of autism. These brain structures normally mediate the processing of emotional and social information, which are the primary characteristics that are disordered in autism.

Cellular abnormalities within the brain are common in autistic patients. Postmortem examinations of autistic human brains show abnormally small, densely packed cells in many areas of the brain including, but not limited to, those illustrated in FIGS. 1A-1C. Abnormally small, densely packed cells suggest that normal development has been curtailed. For example, the programmed cell death, normally mediated by Bc1-2 family genes, has progressed abnormally.

It is also likely that the normal developmental pruning of axons, dendrites, and synapses in the brain of an autistic patient has not occurred at the normal rate. Hence, many autistic patients have an excess number of neural connections within their brain. Excess neural connections may produce aberrant synaptic weighting and global disruption of function within the brain. Moreover, it is believed that, within the overabundance of neural connections in the brain of an autistic patient, many of the neural connections will be faulty and contribute to the disease and its generally intractable symptoms.

It is believed that applying a stimulus to one or more areas of the brain may be useful in treating autistic patients. The stimulus may be used to treat the causes of autism itself and/or any symptom of the disorder (e.g., repetitive behaviors, irritability, tantrums, aggression, impulsivity, and hyperactivity). Consequently, as will be described in more detail below, a stimulator may be implanted in an autistic patient and configured to deliver a stimulus to one or more stimulation sites within the brain. The stimulus may include an electrical stimulation current, one or more drugs, gene infusion, chemical stimulation, thermal stimulation, electromagnetic stimulation, mechanical stimulation, and/or any other suitable stimulation.

As used herein, and in the appended claims, the term “stimulator” will be used broadly to refer to any device that delivers a stimulus, such as an electrical stimulation current, one or more drugs, or other chemical stimulation, thermal stimulation, electromagnetic stimulation, mechanical stimulation, gene infusion, and/or any other suitable stimulation at a stimulation site to treat autism. Thus, the term “stimulator” includes, but is not limited to, a stimulator, microstimulator, implantable pulse generator (IPG), system control unit, cochlear implant, deep brain stimulator, drug pump, or similar device.

The stimulation site referred to herein may include any area within the brain. For example, the stimulation site may include one or more of the following locations within the brain: any area within the temporal lobe (including, but not limited to, the fusiform gyrus) and any area within the limbic system (including, but not limited to, the cingulate gyrus, corpus collosum, thalamus, stria terminalis, caudate nucleus, basal ganglia, hippocampus, entorhinal cortex, amygdala, mammillary body, medial septal nucleus, prefrontal cortex, and fornix). The stimulation site may additionally or alternatively include a cerebral ventricle and/or any area in the frontal lobe, occipital lobe, and parietal lobe.

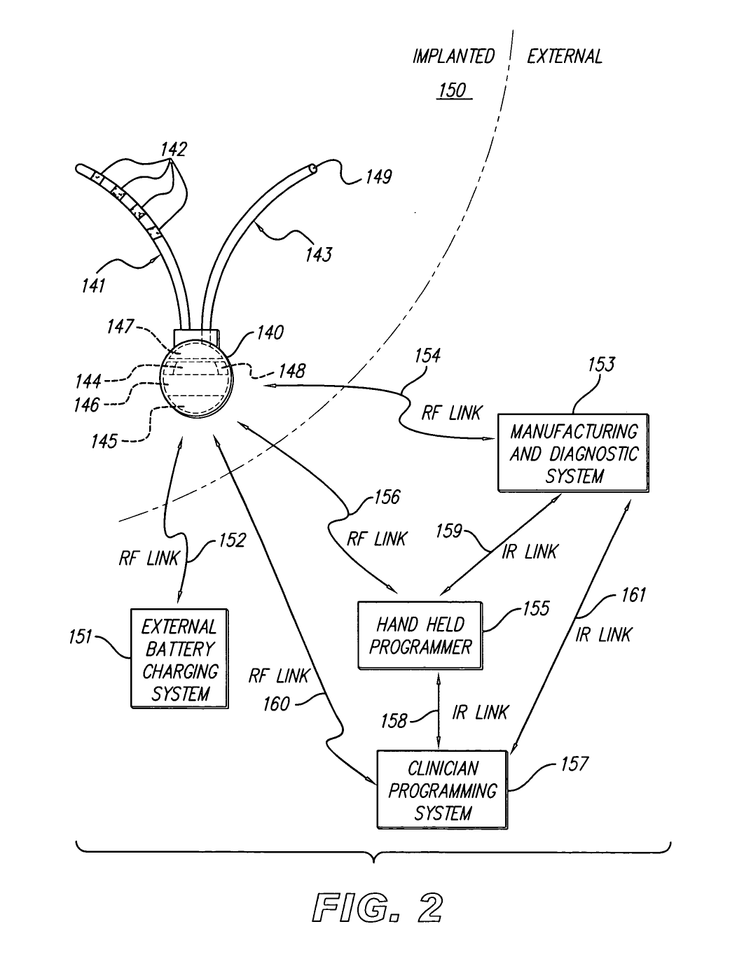

To facilitate an understanding of the methods of optimally treating autism, a more detailed description of the stimulator and its operation will now be given with reference to the figures. FIG. 2 illustrates an exemplary stimulator (140) that may be implanted within a patient (150) and used to apply a stimulus to a stimulation site, e.g., an electrical stimulation of the stimulation site, an infusion of one or more drugs at the stimulation site, or both. The electrical stimulation function of the stimulator (140) will be described first, followed by an explanation of the possible drug delivery function of the stimulator (140). It will be understood, however, that the stimulator (140) may be configured to provide only electrical stimulation, only a drug stimulation, both types of stimulation or any other type of stimulation as best suits a particular patient.

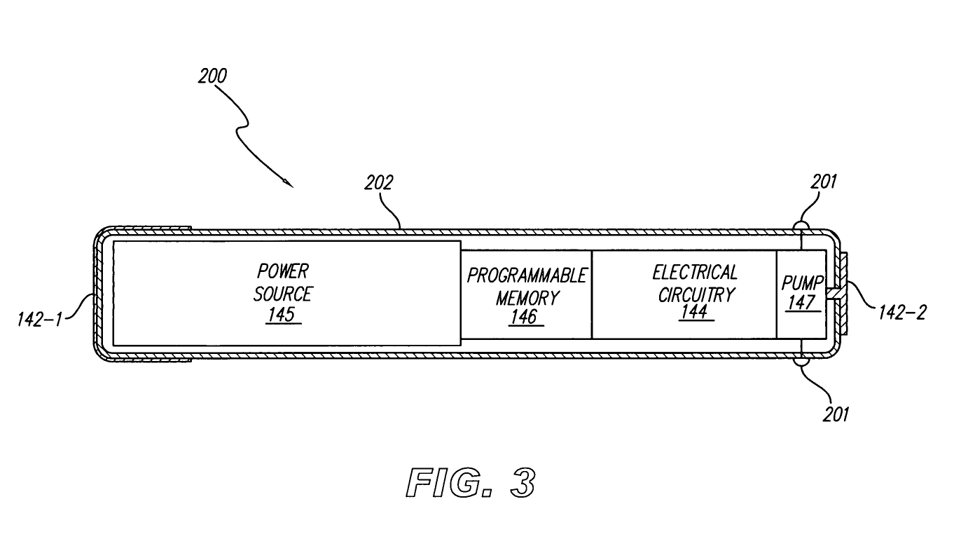

The exemplary stimulator (140) shown in FIG. 2 is configured to provide electrical stimulation to a stimulation site within a patient and may include a lead (141) having a proximal end coupled to the body of the stimulator (140). The lead (141) also includes a number of electrodes (142) configured to apply an electrical stimulation current to a stimulation site. The lead (141) may include any number of electrodes (142) as best serves a particular application. The electrodes (142) may be arranged as an array, for example, having at least two or at least four collinear electrodes. In some embodiments, the electrodes are alternatively inductively coupled to the stimulator (140). The lead (141) may be thin (e.g., less than 3 millimeters in diameter) such that the lead (141) may be positioned near a stimulation site. In some alternative examples, as will be illustrated in connection with FIG. 3, the stimulator (140) is leadless.

As illustrated in FIG. 2, the stimulator (140) includes a number of components. It will be recognized that the stimulator (140) may include additional and/or alternative components as best serves a particular application. A power source (145) is configured to output voltage used to supply the various components within the stimulator (140) with power and/or to generate the power used for electrical stimulation. The power source (145) may be a primary battery, a rechargeable battery, super capacitor, a nuclear battery, a mechanical resonator, an infrared collector (receiving, e.g., infrared energy through the skin), a thermally-powered energy source (where, e.g., memory-shaped alloys exposed to a minimal temperature difference generate power), a flexural powered energy source (where a flexible section subject to flexural forces is part of the stimulator), a bioenergy power source (where a chemical reaction provides an energy source), a fuel cell, a bioelectrical cell (where two or more electrodes use tissue-generated potentials and currents to capture energy and convert it to useable power), an osmotic pressure pump (where mechanical energy is generated due to fluid ingress), or the like. Alternatively, the stimulator (140) may include one or more components configured to receive power from another medical device that is implanted within the patient.

When the power source (145) is a battery, it may be a lithium-ion battery or other suitable type of battery. When the power source (145) is a rechargeable battery, it may be recharged from an external system through a power link such as a radio frequency (RF) power link. One type of rechargeable battery that may be used is described in International Publication WO 01/82398 A1, published Nov. 1, 2001, and/or WO 03/005465 A1, published Jan. 16, 2003, both of which are incorporated herein by reference in their respective entireties. Other battery construction techniques that may be used to make a power source (145) include those shown, e.g., in U.S. Pat. Nos. 6,280,873; 6,458,171, and U.S. Publications 2001/0046625 A1 and 2001/0053476 A1, all of which are incorporated herein by reference in their respective entireties. Recharging can be performed using an external charger.

The stimulator (140) may also include a coil (148) configured to receive and/or emit a magnetic field (also referred to as a radio frequency (RF) field) that is used to communicate with, or receive power from, one or more external devices (151, 153, 155). Such communication and/or power transfer may include, but is not limited to, transcutaneously receiving data from the external device, transmitting data to the external device, and/or receiving power used to recharge the power source (145).

For example, an external battery charging system (EBCS) (151) may provide power used to recharge the power source (145) via an RF link (152). External devices including, but not limited to, a hand held programmer (HHP) (155), clinician programming system (CPS) (157), and/or a manufacturing and diagnostic system (MDS) (153) may be configured to activate, deactivate, program, and test the stimulator (140) via one or more RF links (154, 156). It will be recognized that the links, which are RF links (152, 154, 156) in the illustrated example, may be any type of link used to transmit data or energy, such as an optical link, a thermal link, or any other energy-coupling link. One or more of these external devices (153, 155, 157) may also be used to control the infusion of one or more drugs into the stimulation site.

Additionally, if multiple external devices are used in the treatment of a patient, there may be some communication among those external devices, as well as with the implanted stimulator (140). Again, any type of link for transmitting data or energy may be used among the various devices illustrated. For example, the CPS (157) may communicate with the HHP (155) via an infrared (IR) link (158), with the MDS (153) via an IR link (161), and/or directly with the stimulator (140) via an RF link (160). As indicated, these communication links (158, 161, 160) are not necessarily limited to IR and RF links and may include any other type of communication link. Likewise, the MDS (153) may communicate with the HHP (155) via an IR link (159) or via any other suitable communication link.

The HHP (155), MDS (153), CPS (157), and EBCS (151) are merely illustrative of the many different external devices that may be used in connection with the stimulator (140). Furthermore, it will be recognized that the functions performed by any two or more of the HHP (155), MDS (153), CPS (157), and EBCS (151) maybe performed by a single external device. One or more of the external devices (153, 155, 157) may be embedded in a seat cushion, mattress cover, pillow, garment, belt, strap, pouch, or the like so as to be positioned near the implanted stimulator (140) when in use.

The stimulator (140) may also include electrical circuitry (144) configured to produce electrical stimulation pulses that are delivered to the stimulation site via the electrodes (142). In some embodiments, the stimulator (140) may be configured to produce monopolar stimulation. The stimulator (140) may alternatively or additionally be configured to produce multipolar stimulation including, but not limited to, bipolar or tripolar stimulation.

The electrical circuitry (144) may include one or more processors configured to decode stimulation parameters and generate the stimulation pulses. In some embodiments, the stimulator (140) has at least four channels and drives up to sixteen electrodes or more. The electrical circuitry (144) may include additional circuitry such as capacitors, integrated circuits, resistors, coils, and the like configured to perform a variety of functions as best serves a particular application.

The stimulator (140) may also include a programmable memory unit (146) for storing one or more sets of data and/or stimulation parameters. The stimulation parameters may include, but are not limited to, electrical stimulation parameters, drug stimulation parameters, and other types of stimulation parameters. The programmable memory (146) allows a patient, clinician, or other user of the stimulator (140) to adjust the stimulation parameters such that the stimulation applied by the stimulator (140) is safe and efficacious for treatment of a particular patient. The different types of stimulation parameters (e.g., electrical stimulation parameters and drug stimulation parameters) may be controlled independently. However, in some instances, the different types of stimulation parameters are coupled. For example, electrical stimulation may be programmed to occur only during drug stimulation or vice versa. Alternatively, the different types of stimulation may be applied at different times or with only some overlap. The programmable memory (146) may be any type of memory unit such as, but not limited to, random access memory (RAM), static RAM (SRAM), a hard drive, or the like.

The electrical stimulation parameters may control various parameters of the stimulation current applied to a stimulation site including, but not limited to, the frequency, pulse width, amplitude, waveform (e.g., square or sinusoidal), electrode configuration (i.e., anode-cathode assignment), burst pattern (e.g., burst on time and burst off time), duty cycle or burst repeat interval, ramp on time, and ramp off time of the stimulation current that is applied to the stimulation site. The drug stimulation parameters may control various parameters including, but not limited to, the amount of drugs infused at the stimulation site, the rate of drug infusion, and the frequency of drug infusion. For example, the drug stimulation parameters may cause the drug infusion rate to be intermittent, constant, or bolus. Other stimulation parameters that characterize other classes of stimuli are possible. For example, when tissue is stimulated using electromagnetic radiation, the stimulation parameters may characterize the intensity, wavelength, and timing of the electromagnetic radiation stimuli. When tissue is stimulated using mechanical stimuli, the stimulation parameters may characterize the pressure, displacement, frequency, and timing of the mechanical stimuli.

Specific stimulation parameters may have different effects on different types of autism and/or different patients. Thus, in some embodiments, the stimulation parameters may be adjusted by the patient, a clinician, or other user of the stimulator (140) as best serves the particular autistic patient being treated. The stimulation parameters may also be automatically adjusted by the stimulator (140), as will be described below. For example, the stimulator (140) may increase excitement of a stimulation site by applying a stimulation current having a relatively low frequency (e.g., less than 100 Hz). The stimulator (140) may also decrease excitement of a stimulation site by applying a relatively high frequency (e.g., greater than 100 Hz). The stimulator (140) may also, or alternatively, be programmed to apply the stimulation current to a stimulation site intermittently or continuously.

Additionally, the exemplary stimulator (140) shown in FIG. 2 is configured to provide drug stimulation to an autistic patient by applying one or more drugs at a stimulation site within the brain of the patient. For this purpose, a pump (147) may also be included within the stimulator (140). The pump (147) is configured to store and dispense one or more drugs, for example, through a catheter (143). The catheter (143) is coupled at a proximal end to the stimulator (140) and may have an infusion outlet (149) for infusing dosages of the one or more drugs at the stimulation site. In some embodiments, the stimulator (140) may include multiple catheters (143) and/or pumps (147) for storing and infusing dosages of the one or more drugs at the stimulation site.

The pump (147) or controlled drug release device described herein may include any of a variety of different drug delivery systems. Controlled drug release devices based upon a mechanical or electromechanical infusion pump may be used. In other examples, the controlled drug release device can include a diffusion-based delivery system, e.g., erosion-based delivery systems (e.g., polymer-impregnated with drug placed within a drug-impermeable reservoir in communication with the drug delivery conduit of a catheter), electrodiffusion systems, and the like. Another example is a convective drug delivery system, e.g., systems based upon electroosmosis, vapor pressure pumps, electrolytic pumps, effervescent pumps, piezoelectric pumps and osmotic pumps. Another example is a micro-drug pump.

Exemplary pumps (147) or controlled drug release devices suitable for use as described herein include, but are not necessarily limited to, those disclosed in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,360,019; 4,487,603; 4,627,850; 4,692,147; 4,725,852; 4,865,845; 5,057,318; 5,059,423; 5,112,614; 5,137,727; 5,234,692; 5,234,693; 5,728,396; 6,368,315 and the like. Additional exemplary drug pumps suitable for use as described herein include, but are not necessarily limited to, those disclosed in U.S. Pat. Nos. 4,562,751; 4,678,408; 4,685,903; 5,080,653; 5,097,122; 6,740,072; and 6,770,067. Exemplary micro-drug pumps suitable for use as described herein include, but are not necessarily limited to, those disclosed in U.S. Pat. Nos. 5,234,692; 5,234,693; 5,728,396; 6,368,315; 6,666,845; and 6,620,151. All of these listed patents are incorporated herein by reference in their respective entireties.

The one or more drugs that may be applied to a stimulation site to treat autism may have an excitatory effect on the stimulation site. Additionally or alternatively, the one or more drugs may have an inhibitory effect on the stimulation site to treat autism. Exemplary excitatory drugs that may be applied to a stimulation site to treat autism include, but are not limited to, at least one or more of the following: an excitatory neurotransmitter (e.g., glutamate, dopamine, norepinephrine, epinephrine, acetylcholine, serotonin); an excitatory neurotransmitter agonist (e.g., glutamate receptor agonist, L-aspartic acid, N-methyl-D-aspartic acid (NMDA), bethanechol, norepinephrine); an inhibitory neurotransmitter antagonist(s) (e.g., bicuculline); an agent that increases the level of an excitatory neurotransmitter (e.g., edrophonium, Mestinon); and/or an agent that decreases the level of an inhibitory neurotransmitter (e.g., bicuculline).

Exemplary inhibitory drugs that may be applied to a stimulation site to treat autism include, but are not limited to, at least one or more of the following: an inhibitory neurotransmitter(s) (e.g., gamma-aminobutyric acid, a.k.a. GABA, dopamine, glycine); an agonist of an inhibitory neurotransmitter (e.g., a GABA receptor agonist such as midazolam or clondine, muscimol); an excitatory neurotransmitter antagonist(s) (e.g. prazosin, metoprolol, atropine, benztropine); an agent that increases the level of an inhibitory neurotransmitter; an agent that decreases the level of an excitatory neurotransmitter (e.g., acetylcholinesterase, Group II metabotropic glutamate receptor (mGluR) agonists such as DCG-IV); a local anesthetic agent (e.g., lidocaine); and/or an analgesic medication. It will be understood that some of these drugs, such as dopamine, may act as excitatory neurotransmitters in some stimulation sites and circumstances, and as inhibitory neurotransmitters in other stimulation sites and circumstances.

Additional or alternative drugs that may be applied to a stimulation site to treat autism include at least one or more of the following substances: one or more genes (e.g., NRCAM, LRRN3, KIAA0716, LAMB1, CENTG2) neurotrophic factors (e.g., brain derived neotrophic factors (BDNF) and glial cell line derived neurotrophic factors (GDNF)), steroids, antibiotics, analgesics, opioids (e.g., codeine, oxycodone, propoxyphene), acetaminophen, non-steroidal anti-inflammatory medications (NSAIDS) (e.g., ibuprofen, naproxen, COX-2 inhibitors); corticosteroids (e.g., triamcinolone, hexacetonide, solumedrol), hyaluronic acid derivatives (e.g., hylan G-F 20), colchicines, and hydroxychloroquine.

Any of the drugs listed above, alone or in combination, or other drugs or combinations of drugs developed or shown to treat autism or its symptoms may be applied to the stimulation site to treat autism. In some embodiments, the one or more drugs are infused chronically into the stimulation site. Additionally or alternatively, the one or more drugs may be infused acutely into the stimulation site in response to a biological signal or a sensed need for the one or more drugs.

The stimulator (140) of FIG. 2 is illustrative of many types of stimulators that may be used to apply a stimulus to a stimulation site to treat autism. For example, the stimulator (140) may include an implantable pulse generator (IPG) coupled to one or more leads having a number of electrodes, a spinal cord stimulator (SCS), a cochlear implant, a deep brain stimulator, a drug pump (mentioned previously), a micro-drug pump (mentioned previously), or any other type of implantable stimulator configured to deliver a stimulus at a stimulation site within a patient. Exemplary IPGs suitable for use as described herein include, but are not limited to, those disclosed in U.S. Pat. Nos. 6,381,496, 6,553,263; and 6,760,626. Exemplary spinal cord stimulators suitable for use as described herein include, but are not limited to, those disclosed in U.S. Pat. Nos. 5,501,703; 6,487,446; and 6,516,227. Exemplary cochlear implants suitable for use as described herein include, but are not limited to, those disclosed in U.S. Pat. Nos. 6,219,580; 6,272,382; and 6,308,101. Exemplary deep brain stimulators suitable for use as described herein include, but are not limited to, those disclosed in U.S. Pat. Nos. 5,938,688; 6,016,449; and 6,539,263. All of these listed patents are incorporated herein by reference in their respective entireties.

Alternatively, the stimulator (140) may include an implantable microstimulator, such as a BION® microstimulator (Advanced Bionics® Corporation, Valencia, Calif.). Various details associated with the manufacture, operation, and use of implantable microstimulators are disclosed in U.S. Pat. Nos. 5,193,539; 5,193,540; 5,312,439; 6,185,452; 6,164,284; 6,208,894; and 6,051,017. All of these listed patents are incorporated herein by reference in their respective entireties.

FIG. 3 illustrates an exemplary microstimulator (200) that may be used as the stimulator (140; FIG. 2) described herein. Other configurations of the microstimulator (200) are possible, as shown in the above-referenced patents and as described further below.

As shown in FIG. 3, the microstimulator (200) may include the power source (145), the programmable memory (146), the electrical circuitry (144), and the pump (147) described in connection with FIG. 2. These components are housed within a capsule (202). The capsule (202) may be a thin, elongated cylinder or any other shape as best serves a particular application. The shape of the capsule (202) may be determined by the structure of the desired target nerve, the surrounding area, and the method of implantation. In some embodiments, the volume of the capsule (202) is substantially equal to or less than three cubic centimeters. In some embodiments, the microstimulator (200) may include two or more leadless electrodes (142) disposed on the outer surface of the microstimulator (200).

The external surfaces of the microstimulator (200) may advantageously be composed of biocompatible materials. For example, the capsule (202) may be made of glass, ceramic, metal, or any other material that provides a hermetic package that will exclude water vapor but permit passage of electromagnetic fields used to transmit data and/or power. The electrodes (142) may be made of a noble or refractory metal or compound, such as platinum, iridium, tantalum, titanium, titanium nitride, niobium or alloys of any of these, in order to avoid corrosion or electrolysis which could damage the surrounding tissues and the device.

The microstimulator (200) may also include one or more infusion outlets (201). The infusion outlets (201) facilitate the infusion of one or more drugs at a stimulation site to treat autism. The infusion outlets (201) may dispense one or more drugs directly to the treatment site. Alternatively, catheters may be coupled to the infusion outlets (201) to deliver the drug therapy to a treatment site some distance from the body of the microstimulator (200). The stimulator (200) of FIG. 3 also includes electrodes (142-1 and 142-2) at either end of the capsule (202). One of the electrodes (142) may be designated as a stimulating electrode to be placed close to the treatment site and one of the electrodes (142) may be designated as an indifferent electrode used to complete a stimulation circuit.

The microstimulator (200) may be implanted within a patient with a surgical tool such as a hypodermic needle, bore needle, or any other tool specially designed for the purpose. Alternatively, the microstimulator (200) may be implanted using endoscopic or laparoscopic techniques.

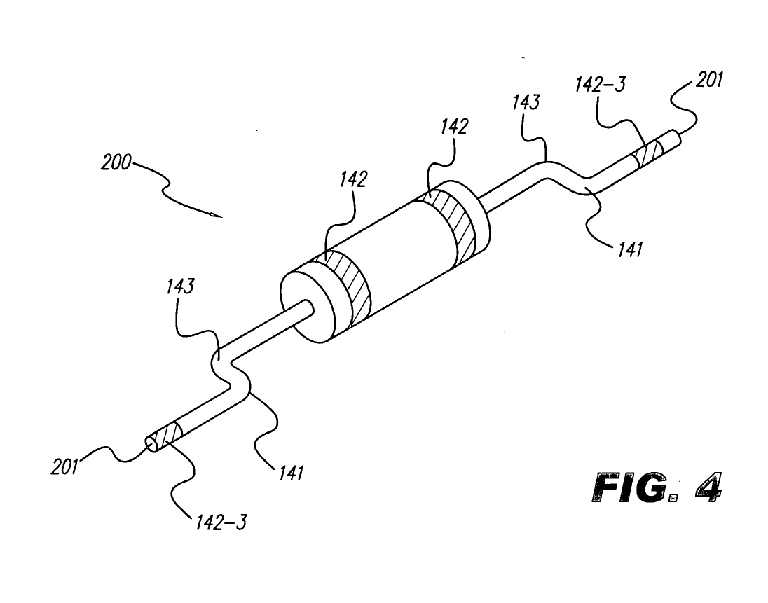

FIG. 4 shows an example of a microstimulator (200) with one or more catheters (143) coupled to the infusion outlets on the body of the microstimulator (200). With the catheters (143) in place, the infusion outlets (201) that actually deliver the drug therapy to target tissue are located at the ends of catheters (143). Thus, in the example of FIG. 4, a drug therapy is expelled by the pump (147, FIG. 3) from an infusion outlet (201, FIG. 3) in the casing (202, FIG. 3) of the microstimulator (200), through the catheter (143), out an infusion outlet (201) at the end of the catheter (143) to the stimulation site within the patient. As shown in FIG. 4, the catheters (143) may also serve as leads (141) having one or more electrodes (142-3) disposed thereon. Thus, the catheters (143) and leads (141) of FIG. 4 permit infused drugs and/or electrical stimulation current to be directed to a stimulation site while allowing most elements of the microstimulator (200) to be located in a more surgically convenient site. The example of FIG. 4 may also include leadless electrodes (142) disposed on the housing of the microstimulator (200), in the same manner described above.

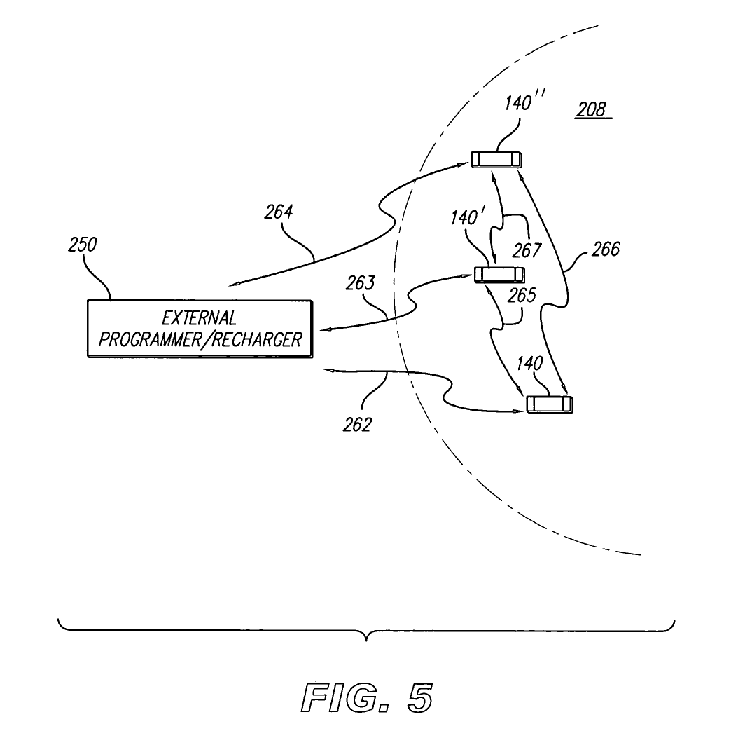

Returning to FIG. 2, the stimulator (140) may be configured to operate independently. Alternatively, as shown in FIG. 5 and described in more detail below, the stimulator (140) may be configured to operate in a coordinated manner with one or more additional stimulators, other implanted devices, or other devices external to the patient's body. For instance, a first stimulator may control, or operate under the control of, a second stimulator, other implanted device, or other device external to the patient's body. The stimulator (140) may be configured to communicate with other implanted stimulators, other implanted devices, or other devices external to the patient's body via an RF link, an untrasonic link, an optical link, or any other type of communication link. For example, the stimulator (140) may be configured to communicate with an external remote control unit that is capable of sending commands and/or data to the stimulator (140) and that is configured to receive commands and/or data from the stimulator (140).

In order to determine the strength and/or duration of electrical stimulation and/or amount and/or type(s) of stimulating drug(s) required to most effectively treat autism, various indicators of autism and/or a patient's response to treatment may be sensed or measured. These indicators include, but are not limited to, electrical activity of the brain (e.g., EEG); neurotransmitter levels; hormone levels; metabolic activity in the brain; blood flow rate in the head, neck or other areas of the body; medication levels within the patient; patient or caregiver input, e.g., the stimulation may be in response to a temper tantrum or other physical manifestation of autism; temperature of tissue at the stimulation site; physical activity level, e.g. based on accelerometer recordings; and/or brain hyperexcitability, e.g. increased response of given tissue to the same input. In some embodiments, the stimulator (140) may be configured to adjust the stimulation parameters in a closed loop manner in response to these measurements. The stimulator (140) may be configured to perform the measurements. Alternatively, other sensing devices may be configured to perform the measurements and transmit the measured values to the stimulator (140). Exemplary sensing devices include, but are not limited to, chemical sensors, electrodes, optical sensors, mechanical (e.g., motion, pressure) sensors, and temperature sensors.

Thus, one or more external devices may be provided to interact with the stimulator (140), and may be used to accomplish at least one or more of the following functions:

Function 1: If necessary, transmit electrical power to the stimulator (140) in order to power the stimulator (140) and/or recharge the power source (145).

Function 2: Transmit data to the stimulator (140) in order to change the stimulation parameters used by the stimulator (140).

Function 3: Receive data indicating the state of the stimulator (140) (e.g., battery level, drug level, stimulation parameters, etc.).

Additional functions may include adjusting the stimulation parameters based on information sensed by the stimulator (140) or by other sensing devices.

By way of example, an exemplary method of treating an autistic patient may be carried out according to the following sequence of procedures. The steps listed below may be modified, reordered, and/or added to as best serves a particular application.

1. A stimulator (140) is implanted so that its electrodes (142) and/or infusion outlet (149) are coupled to or located near a stimulation site (e.g., a location within the limbic system). If the stimulator (140) is a microstimulator, such as the microstimulator (200) described in FIG. 3, the microstimulator itself may be coupled to the stimulation site.

2. The stimulator (140) is programmed to apply at least one stimulus to the stimulation site. The stimulus may include electrical stimulation, drug stimulation, gene infusion, chemical stimulation, thermal stimulation, electromagnetic stimulation, mechanical stimulation, and/or any other suitable stimulation.

3. When the patient desires to invoke stimulation, the patient sends a command to the stimulator (140) (e.g., via a remote control) such that the stimulator (140) delivers the prescribed stimulation. The stimulator (140) may be alternatively or additionally configured to automatically apply the stimulation in response to sensed indicators of autism.

4. To cease stimulation, the patient may turn off the stimulator (140) (e.g., via a remote control).

5. Periodically, the power source (145) of the stimulator (140) is recharged, if necessary, in accordance with Function 1 described above. As will be described below, this recharging function can be made much more efficient using the principles disclosed herein.

In other examples, the treatment administered by the stimulator (140), i.e., drug therapy and/or electrical stimulation, may be automatic and not controlled or invoked by the patient.

For the treatment of different patients, it may be desirable to modify or adjust the algorithmic functions performed by the implanted and/or external components, as well as the surgical approaches. For example, in some situations, it may be desirable to employ more than one stimulator (140), each of which could be separately controlled by means of a digital address. Multiple channels and/or multiple patterns of stimulation may thereby be used to deal with the multiple medical conditions, such as, for example, the combination of autism with a seizure disorder.

As shown in the example of FIG. 5, a first stimulator (140) implanted beneath the skin of the patient (208) provides a stimulus to a first location; a second stimulator (140′) provides a stimulus to a second location; and a third stimulator (140″) provides a stimulus to a third location. As mentioned earlier, the implanted devices may operate independently or may operate in a coordinated manner with other implanted devices or other devices external to the patient's body. That is, an external controller (250) may be configured to control the operation of each of the implanted devices (140, 140′, and 140″). In some embodiments, an implanted device, e.g. stimulator (140), may control, or operate under the control of, another implanted device(s), e.g. stimulator (140′) and/or stimulator (140″). Control lines (262-267) have been drawn in FIG. 5 to illustrate that the external controller (250) may communicate or provide power to any of the implanted devices (140, 140′, and 140″) and that each of the various implanted devices (140, 140′, and 140″) may communicate with and, in some instances, control any of the other implanted devices.

As a further example of multiple stimulators (140) operating in a coordinated manner, the first and second stimulators (140, 140′) of FIG. 5 may be configured to sense various indicators of autism and transmit the measured information to the third stimulator (140″). The third stimulator (140″) may then use the measured information to adjust its stimulation parameters and apply stimulation to a stimulation site accordingly. The various implanted stimulators may, in any combination, sense indicators of autism, communicate or receive data on such indicators, and adjust stimulation parameters accordingly.

Alternatively, the external device (250) or other external devices communicating with the external device may be configured to sense various indicators of a patient's condition. The sensed indicators can then be collected by the external device (250) for relay to one or more of the implanted stimulators or may be transmitted directly to one or more of the implanted stimulators by any of an array of external sensing devices. In either case, the stimulator, upon receiving the sensed indicator(s), may adjust stimulation parameters accordingly. In other examples, the external controller (250) may determine whether any change to stimulation parameters is needed based on the sensed indicators. The external device (250) may then signal a command to one or more of the stimulators to adjust stimulation parameters accordingly.

The stimulator (140) of FIG. 2 may be implanted within an autistic patient using any suitable surgical procedure such as, but not limited to, injection, small incision, open placement, laparoscopy, or endoscopy. Exemplary methods of implanting a microstimulator, for example, are described in U.S. Pat. Nos. 5,193,539; 5,193,540; 5,312,439; 6,185,452; 6,164,284; 6,208,894; and 6,051,017. Exemplary methods of implanting an SCS, for example, are described in U.S. Pat. Nos. 5,501,703; 6,487,446; and 6,516,227. Exemplary methods of implanting a deep brain stimulator, for example, are described in U.S. Pat. Nos. 5,938,688; 6,016,449; and 6,539,263. All of these listed patents are incorporated herein by reference in their respective entireties.

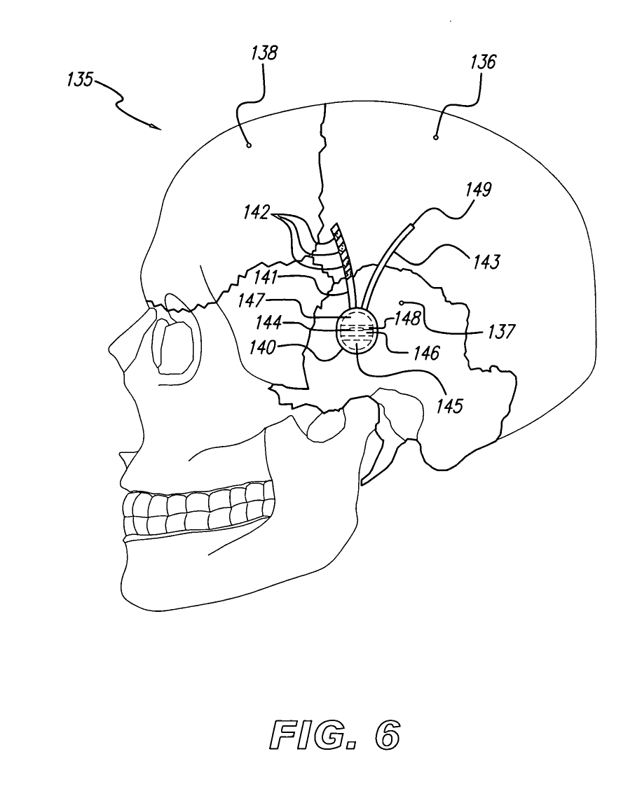

By way of example, FIG. 6 shows a stimulator (140) (e.g., a deep brain stimulator) that has been implanted beneath the scalp of a patient to stimulate a stimulation site within the brain associated with autism. The stimulator (140) may be implanted in a surgically-created shallow depression or opening in the skull (135). For instance, the depression may be made in the parietal bone (136), temporal bone (137), frontal bone (138), or any other bone within the skull (135) as best serves a particular application. The stimulator (140) may conform to the profile of surrounding tissue(s) and/or bone(s), thereby minimizing the pressure applied to the skin or scalp. Additionally or alternatively, the stimulator (140) may be implanted in a subdural space over any of the lobes of the brain, in a sinus cavity, or in an intracerebral ventricle.

In some embodiments, as shown in FIG. 6, a lead (141) and/or catheter (143) may run subcutaneously to an opening in the skull (135) and pass through the opening into or onto a stimulation site in the brain. Alternatively, the stimulator (140) is leadless and is configured to generate a stimulus that passes through the skull. In this manner, the brain may be stimulated without having to physically invade the brain itself.

In some examples, the stimulation applied by the stimulator (140) is configured to activate inactive regions of the brain that are associated with autism. For example, the stimulation may be configured to activate one or more areas in the limbic system to treat autism. The stimulation may additionally or alternatively be configured to treat autism by promoting neurotransmission along nerve axons that innervate various regions of the brain.

As mentioned, many autistic patients have an excess number of neural connections and/or faulty neural connections within their brain. Hence, it is believed that autism may be treated by inducing neural remodeling to remove and/or repair faulty neural connections in the brain that are responsible for autism. As used herein and in the appended claims, unless otherwise specifically denoted, neural remodeling is the ability of neural circuits to undergo changes in function or organization. In some examples, the stimulus applied by the stimulator (140) is configured to induce neural remodeling to return neural structures within the brain to a juvenile neural phenotype. Developmental events will then recur naturally or with the aid of stimuli, thereby allowing a normal adult phenotype to be established.

In some examples, the stimulus applied by the stimulator includes electroconvulsive therapy and/or pentylenetetrazol injections. These types of stimulation cause global seizure activity, which in turn induces neural remodeling. The stimulus may additionally or alternatively include one or more drugs, genes, or other substances that support neural remodeling of cellular connections. These substances may include, but are not limited to, neurotrophic factors, fibroblast growth factors, ethanol, steroid hormones such as testosterone, and/or any other drug listed herein. Injections of biologic or genetic material may induce neural remodeling through upregulating proapoptotic genes and/or proteins of the Bc1-2 family such as Bax or Bid, upregulating gap junction proteins, and knocking down expression and translation of actin and microtubule proteins to induce pruning of dendrites, axons, and synapses.

The preceding description has been presented only to illustrate and describe embodiments of the invention. It is not intended to be exhaustive or to limit the invention to any precise form disclosed. Many modifications and variations are possible in light of the above teaching.

What is claimed is:

1. A method of treating autism, said method comprising:
applying at least one stimulus with an implanted stimulator to a stimulation site within a brain of an autistic patient;
wherein said stimulus is in accordance with one or more stimulation parameters and configured to treat autism.

2. The method of claim 1, wherein said stimulation site comprises at least one or more of a location within a temporal lobe, cerebral ventricle, prefrontal cortex, and a location within a limbic system of said patient.

3. The method of claim 1, wherein said stimulator is coupled to one or more electrodes, and wherein said stimulus comprises a stimulation current delivered via said electrodes.

4. The method of claim 1, wherein said stimulus comprises one or more drugs delivered to said stimulation site.

5. The method of claim 1, wherein said stimulus comprises a stimulation current delivered to said stimulation site and one or more drugs delivered to said stimulation site.

6. The method of claim 1, further comprising inducing neural remodeling within said brain with said stimulus to treat said autism.

7. The method of claim 1, further comprising sensing at least one indicator related to said autism and using said at least one sensed indicator to adjust one or more of said stimulation parameters.

8. The method of claim 8, wherein said at least one indicator comprises at least one or more of an electrical activity of said brain, a chemical level of said brain, a neurotransmitter level, a hormone level, and a medication level.

9. The method of claim 1, wherein said stimulator is implanted within at least one or more of a subdural space, a sinus cavity, and a cerebral ventricle.

10. A system for treating autism, said system comprising:
a stimulator configured to apply at least one stimulus to a stimulation site within a brain of an autistic patient in accordance with one or more stimulation parameters;
wherein said stimulation parameters and resulting stimulus are configured to treat said autism.

11. The system of claim 10, wherein said stimulation site comprises at least one or more of a location within a temporal lobe, cerebral ventricle, prefrontal cortex, and a location within a limbic system of said patient.

12. The system of claim 10, wherein said stimulator is coupled to one or more electrodes, and wherein said stimulus comprises a stimulation current delivered via said electrodes.

13. The system of claim 10, wherein said stimulus comprises one or more drugs delivered to said stimulation site.

14. The system of claim 10, wherein said stimulus comprises a stimulation current delivered to said stimulation site and one or more drugs delivered to said stimulation site.

15. The system of claim 10, wherein said stimulator is configured to induce neural remodeling within said brain to treat said autism.

16. The system of claim 10, further comprising:
a sensor device for sensing at least one indicator related to said autism;
wherein said stimulator uses said at least one sensed indicator to adjust one or more of said stimulation parameters.

17. The system of claim 16, wherein said at least one indicator comprises at least one or more of an electrical activity of said brain, a chemical level of said brain, a neurotransmitter level, a hormone level, and a medication level.

18. The system of claim 10, wherein said stimulator is implanted within at least one or more of a subdural space and a cerebral ventricle.

19. A system for treating autism, said system comprising:
means for applying at least one stimulus to a stimulation site within a brain of an autistic patient in accordance with one or more stimulation parameters; and
means for adjusting said stimulation parameters such that said stimulus is effective to treat said autism.

20. The system of claim 19, wherein said stimulus comprises at least one or more of a stimulation current and one or more drugs delivered to said stimulation site.
